(19) United States
(12) Patent Application Publication
(10) Pub. No.: US 2023/0065958 A1
(43) Pub. Date: Mar. 2, 2023

(54) DYNAMIC THREE-DIMENSIONAL MERIDIAN SYSTEM OF HUMAN BODY

(71) Applicant: China Medical University, Taichung City (TW)

(72) Inventors: Jaung Geng Lin, Taipei City (TW); Shung Te Kao, Taichung City (TW); Yu Chen Lee, Taichung City (TW); Ming Cheng Huang, Taichung City (TW)

(73) Assignee: China Medical University, Taichung City (TW)

(21) Appl. No.: 17/893,322

(22) Filed: Aug. 23, 2022

(30) Foreign Application Priority Data
Aug. 24, 2021 (TW) 110131309

(51) Int. Cl. G06F 3/04815; G06T 15/00; G06T 19/20; G06F 3/0488

(52) U.S. Cl. CPC G06F 3/04815; G06T 15/00; G06T 19/20; G06F 3/0488; G06T 2200/24; G06T 2210/41; G06T 2200/04; G06T 2219/2016

(57) ABSTRACT

A dynamic three-dimensional meridian system of a human body includes a non-transitory machine readable medium, a touch-screen monitor and a processor. The non-transitory machine readable medium includes a human meridian program, a three-dimensional anatomical image database of the human body and a meridian database. The human meridian program includes a three-dimensional image establishing module, a human meridian building module and an operating module. The touch-screen monitor is signally connected to the non-transitory machine readable medium, wherein the touch-screen monitor is for displaying a dynamic three-dimensional meridian image of the human body and a graphical user interface of the human meridian program. The processor is signally connected to the non-transitory machine readable medium and the touch-screen monitor.

### RELATED APPLICATIONS

This application claims priority to Taiwan Application Serial Number 110131309, filed Aug. 24, 2021, which is herein incorporated by reference.

### BACKGROUND

### Technical Field

The present disclosure relates to a human meridian displaying system. More particularly, the present disclosure relates to a dynamic three-dimensional meridian system of a human body which can show images dynamically and can be interactive with a user.

### Description of Related Art

In the point of view of the traditional Chinese medicine, the meridian system is composed of a plurality of channels that are for the circulation of qi and blood and connect among the organs, the body surface and all parts of the body, and thus the meridian system is an important system for signal transduction and functional regulation in the human body. For more than 2,000 years, the meridian system has been applied for human health maintenance, disease prevention, physical examination, clinical treatment, related health education, etc. Accordingly, by the methods of pressing or acupuncture to stimulate the acupoints of the meridian system of the human body, it is favorable for diagnosing and treating of the corresponding diseases.

The bronze acupuncture figure is often used as an acupuncture point model in current Chinese medicine teaching so as to intuitively illustrate the types of the meridians and the locations of the acupoints thereof in the meridian system of the human body. In detail, fourteen meridians formed by the connection of many acupoints are shown on the bronze acupuncture figure, wherein the fourteen meridians respectively belong to the “twelve primary meridians” and “Conception and Governor vessels”, so that the bronze acupuncture figure can be used to illustrate the details of the channel theory so as to facilitate understanding the principles of the science of acupuncture and moxibustion. However, the conventional bronze acupuncture figure is usually made of metal or plastic material, so that not only the bronze acupuncture figure will occupy a certain space, but also the weight thereof also cannot be underestimated. Thus, the teaching related to the meridian system by using the bronze acupuncture figure will be performed under the restriction of the position of the bronze acupuncture figure, resulting in the inconvenience in the use thereof. Furthermore, the conventional bronze acupuncture figure should be used along with the textbook so as to understand the circulating direction of each meridian and the depth of acupoints, and thus there are many limitations in the teaching effectiveness thereof.

Along with the advance in technology, the use of computer technology to improve the meridian model of the human body in Chinese medicine is also increasingly advanced. The common meridian wall charts, book illustrations and patterns in the web page are mostly produced by the computer graphic drawing technology. Further, two-dimensional animations are also used to present the circulation condition, the circulation paths and the distribution area in the human body of the meridians. Furthermore, there is a device which shows the meridian system by a three-dimensional graphic in the current market, and the device is also applied to the related teaching areas. However, the aforementioned device for presenting the meridian system and the acupoints by the current three-dimensional technology only shows the meridians and the related acupoints on a three-dimensional mannequin. Thus, not only there is unable to operate or dismantle the anatomical structures, the meridians and the acupoints of the three-dimensional mannequin, but also the dynamic changes of the circulation condition in the meridians between the human tissues and the acupoints are impossible to be visualized.

Therefore, there is an urgent need to develop a meridian display system that can more clearly show the circulation direction of each meridian and the details of acupoints so as to facilitate the teaching and the development of traditional Chinese medicine.

### SUMMARY

According to one aspect of the present disclosure, a dynamic three-dimensional meridian system of a human body includes a non-transitory machine readable medium, a touch-screen monitor and a processor. The non-transitory machine readable medium includes a human meridian program, a three-dimensional anatomical image database of the human body and a meridian database, wherein the three-dimensional anatomical image database of the human body includes a plurality of three-dimensional anatomical image data of the human body, and the meridian database includes a plurality of acupoint data and a plurality of meridian circulation data. The human meridian program includes a three-dimensional image establishing module, a human meridian building module and an operating module. The three-dimensional image establishing module is for processing the plurality of three-dimensional anatomical image data of the human body so as to obtain a simulated three-dimensional image of the human body. The human meridian building module is for integrating the plurality of acupoint data, the plurality of meridian circulation data and the simulated three-dimensional image of the human body so as to obtain a dynamic three-dimensional meridian image of the human body, wherein the dynamic three-dimensional meridian image of the human body includes a plurality of acupoint location information, a plurality of acupoint depth-location information and a plurality of meridian distribution information. The operating module is for receiving an image operating instruction so as to adjust an image displaying condition of the dynamic three-dimensional meridian image of the human body. The touch-screen monitor is signally connected to the non-transitory machine readable medium, wherein the touch-screen monitor is for displaying the dynamic three-dimensional meridian image of the human body and a graphical user interface of the human meridian program. The processor is signally connected to the non-transitory machine readable medium and the touch-screen monitor, wherein the human meridian program is performed by the processor. The plurality of acupoint location information respectively correspond to a plurality of acupoint location sites which are shown on the dynamic three-dimensional meridian image of the human body, and the plurality of meridian distribution information are established based on actual depths of acupoints of the plurality of acupoint data corresponding thereto and in a circulation order thereof.

### BRIEF DESCRIPTION OF THE DRAWINGS

The present disclosure can be more fully understood by reading the following detailed description of the embodiment, with reference made to the accompanying drawings as follows:

FIG. 1 is a block diagram of a dynamic three-dimensional meridian system of a human body according to one embodiment of the present disclosure.

FIG. 2 is a block diagram of a dynamic three-dimensional meridian system of a human body according to another embodiment of the present disclosure.

FIG. 3 is a schematic view of a dynamic three-dimensional meridian system of a human body of the present disclosure.

FIG. 4 is another schematic view of the dynamic three-dimensional meridian system of the human body of FIG. 3.

FIG. 5 is a schematic view of a touch-screen monitor of the dynamic three-dimensional meridian system of the human body of FIG. 3 for displaying an acupoint data.

FIG. 6 is a schematic view of the touch-screen monitor of the dynamic three-dimensional meridian system of the human body of FIG. 3 for displaying a meridian circulation video file.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

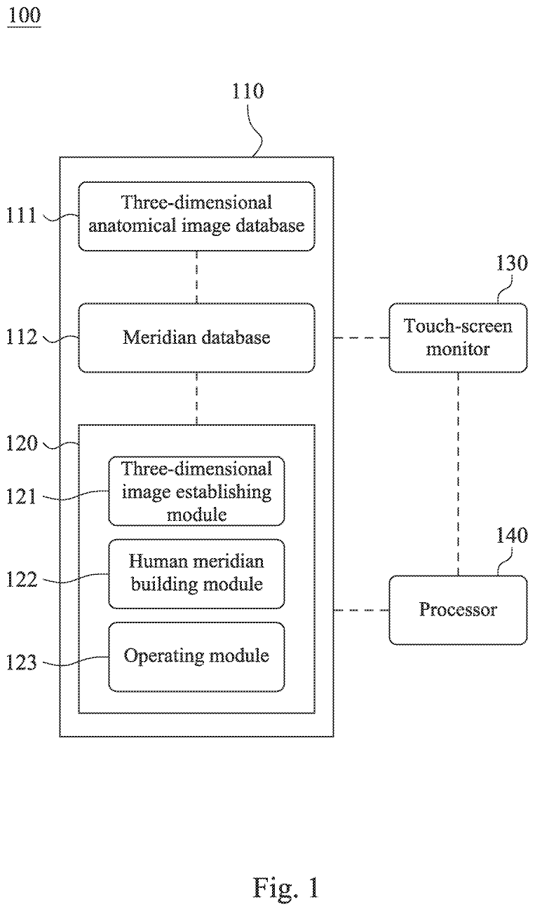

Please refer to FIG. 1, which is a block diagram of a dynamic three-dimensional meridian system 100 of a human body according to one embodiment of the present disclosure. The dynamic three-dimensional meridian system 100 of the human body includes a non-transitory machine readable medium 110, a touch-screen monitor 130 and a processor 140.

The non-transitory machine readable medium 110 includes a human meridian program 120, a three-dimensional anatomical image database 111 of the human body and a meridian database 112, wherein the three-dimensional anatomical image database 111 of the human body includes a plurality of three-dimensional anatomical image data of the human body, and the meridian database 112 includes a plurality of acupoint data and a plurality of meridian circulation data. The non-transitory machine readable medium 110 can be disposed in a computer, a smart phone or other electronic devices that can support the performance of the touch-screen monitor 130. The aforementioned three-dimensional anatomical image data of the human body can be computed tomography (CT) images and can be a three-dimensional anatomical image data of a male human body or a three-dimensional anatomical image data of a female human body, so that the structures of the male human body or the female human body can be presented specifically.

The acupoint data and the meridian circulation data of the meridian database 112 are obtained according to the information about meridians and acupoints disclosed in the book published by the lecturing professor Jaung-Geng Lin, so that the medical application potential of the dynamic three-dimensional meridian system 100 of the human body can be enhanced under the premise of the correct information disclosure. In particular, the meridian circulation data can include the twelve primary meridians circulation data and the eight extraordinary vessels circulation data, a total of twenty kinds of meridian information. The twelve primary meridians circulation data includes Lung (LU) meridian circulation data, Large Intestine (LI) meridian circulation data, Stomach (ST) meridian circulation data, Spleen (SP) meridian circulation data, Heart (HT) meridian circulation data, Small Intestine (SI) meridian circulation data, Bladder (BL) meridian circulation data, Kidney (KI) meridian circulation data, Pericardium (PC) meridian circulation data, Triple Energizer (TE) meridian circulation data, Gallbladder (GB) meridian circulation data and Liver (LV) meridian circulation data. The eight extraordinary vessels circulation data includes Dumai (GV) meridian circulation data, Renmai (CV) meridian circulation data, Chongmai (TV) meridian circulation data, Daimai (BV) meridian circulation data, Yinweimai (YinLV) meridian circulation data, Yangweimai (YangLV) meridian circulation data, Yinqiaomai (YinHV) meridian circulation data and Yangqiaomai (YangHV) meridian circulation data. Further, the details of the aforementioned meridian circulation data are the well-known professional medical knowledge in the technical field of the present invention, so the relevant information thereof will not be described herein.

The human meridian program 120 includes a three-dimensional image establishing module 121, a human meridian building module 122 and an operating module 123. In particular, the human meridian program 120 can be a touch program of multi-point interaction for a single user so as to present the teaching and the application thereof more completely, but the present disclosure is not limited thereto.

The three-dimensional image establishing module 121 is for processing the plurality of three-dimensional anatomical image data of the human body so as to obtain a simulated three-dimensional image of the human body (not shown). In detail, the simulated three-dimensional image of the human body is established based on the three-dimensional anatomical image data of the human body, wherein those three-dimensional anatomical image data of the human body include the detailed image information about the skin, the bones, nerves, the digestive system, the vascular structure, the respiratory system, the muscles, the urinary system, and the lymphatic system of the human body. Thus, the simulated three-dimensional image of the human body established by the three-dimensional image establishing module 121 will include detailed information on human anatomy so as to facilitate the following applications.

The human meridian building module 122 is for integrating the plurality of acupoint data, the plurality of meridian circulation data and the simulated three-dimensional image of the human body so as to obtain a dynamic three-dimensional meridian image of the human body (not shown), wherein the dynamic three-dimensional meridian image of the human body includes a plurality of acupoint location information, a plurality of acupoint depth-location information and a plurality of meridian distribution information. The plurality of acupoint location information respectively correspond to a plurality of acupoint location sites which are shown on the dynamic three-dimensional meridian image of the human body, and the plurality of meridian distribution information are established based on actual depths of acupoints of the plurality of acupoint data corresponding thereto and in a circulation order thereof. In detail, the human meridian building module 122 will integrate the acupoint data and the meridian circulation data with the simulated three-dimensional image of the human body including the detailed information on human anatomy so as to establish the dynamic three-dimensional meridian image of the human body of the present disclosure. Furthermore, the dynamic three-dimensional meridian image of the human body can further include a bone composition information, an organ distribution information, a blood vessel distribution information, a lymphatic distribution information, a nerve distribution information and a skin condition information, and the acupoint location information, the acupoint depth-location information and the meridian distribution information can be presented with the bone composition information, the organ distribution information, the blood vessel distribution information, the lymphatic distribution information, the nerve distribution information and the skin condition information correspondingly and integrally. Therefore, it is favorable for obtaining the dynamic and realistic visual presenting effects of the meridians along the order of the acupoints and based on the actual depths thereof under the premise of high precision, so that the needs of medical education and medical research can be satisfied.

The operating module 123 is for receiving an image operating instruction so as to adjust an image displaying condition of the dynamic three-dimensional meridian image of the human body. In detail, the image operating instruction can be output by the processor 140 when the user operates the touch-screen monitor 130, and then the image operating instruction will be transmitted to the operating module 123 so as to perform the following actions.

The touch-screen monitor 130 is signally connected to the non-transitory machine readable medium 110, wherein the touch-screen monitor 130 is for displaying the dynamic three-dimensional meridian image of the human body and a graphical user interface of the human meridian program 120. The graphical user interface can include a plurality of touch icons (not shown) and a plurality of functional listings (not shown), the touch icons and the functional listings can be respectively disposed on a first side portion (not shown) and a second side portion (not shown) of the touch-screen monitor 130. In detail, the touch icons can form a first interface arrangement unit, the functional listings can form a second interface arrangement unit, and the first interface arrangement unit and the second interface arrangement unit can be displayed or hidden by being dragged left or right, but the present disclosure is not limited thereto. The details of the touch icons and the functional listings will be described in the following description.

The processor 140 is signally connected to the non-transitory machine readable medium 110 and the touch-screen monitor 130, and the human meridian program 120 is performed by the processor 140. In particular, the processor 140 can be arranged in a computer or an electronic device. The processor 140 can be integrated with the non-transitory machine readable medium 110 in the same computer or the same electronic device, or can be arranged separately from the non-transitory machine readable medium 110, but the present disclosure is not limited thereto.

In detail, in the dynamic three-dimensional meridian system 100 of the human body of the present disclosure, the image operating instruction can include a zooming sub-instruction, an angle adjusting sub-instruction and an image contrast adjusting sub-instruction, and the image displaying condition can include an image displaying size, an image displaying angle and an image contrast. The operating module 123 adjusts the image displaying size, the image displaying angle or the image contrast of the dynamic three-dimensional meridian image of the human body based on the zooming sub-instruction, the angle adjusting sub-instruction or the image contrast adjusting sub-instruction. Further, the user can adjust the size or the presenting angle of the dynamic three-dimensional meridian image of the human body by tapping the zooming icon or the angle adjusting icon of the graphical user interface of the human meridian program 120, or by pinching open or closed with two fingers to zoom in, zoom out or rotate the dynamic three-dimensional meridian image of the human body. Thus, it is favorable for adjusting the size of the dynamic three-dimensional meridian image of the human body or rotating it at 360 degrees angle, so that the acupoint location information, the acupoint depth-location information and the meridian distribution information of the dynamic three-dimensional meridian image of the human body can be displayed in a variety of ways with different proportions and angles.

Furthermore, in the dynamic three-dimensional meridian system 100 of the human body of the present disclosure, the image operating instruction can include an image-presenting adjusting sub-instruction, and the operating module 123 shows at least one of the bone composition information, the organ distribution information, the blood vessel distribution information, the lymphatic distribution information, the nerve distribution information and the skin condition information of the dynamic three-dimensional meridian image of the human body based on the image-presenting adjusting sub-instruction. Accordingly, the detailed information about the acupoint location information, the acupoint depth-location information and the meridian distribution information corresponding to the human anatomical structure can be presented according to the actual needs.

Furthermore, in the dynamic three-dimensional meridian system 100 of the human body of the present disclosure, each of the acupoint data can include an acupoint name information, an acupoint dropping point information, an acupoint selecting method information, an indication information and an acupuncture depth information, and the meridian circulation data can include a plurality of meridian circulation video files. The operating module 123 can receive an acupoint presenting instruction to display one of the plurality of acupoint data on the touch-screen monitor 130, and can receive an animation playing instruction to display one of the meridian circulation video files on the touch-screen monitor 130. Therefore, it is favorable for dynamically displaying the paths and the distribution in the human body of the circulation and the flowing of the meridians, which is beneficial to the subsequent applications.

Furthermore, in the dynamic three-dimensional meridian system 100 of the human body of the present disclosure, one of the meridian circulation video files is displayed on the touch-screen monitor 130 in a sub-window, and the acupoint location information, the acupoint depth-location information and the meridian distribution information are simultaneously displayed on the touch-screen monitor 130. In detail, the twelve primary meridians and the eight extraordinary vessels are circulated in the human endlessly, and each of the meridians has its own circulating time, so when one of the meridian circulation video files is played in the sub-window, the acupoint location information, the acupoint depth-location information and the meridian distribution information can be simultaneously displayed on the touch-screen monitor 130. Thus, it is favorable for presenting the meridian circulation data clearly.

Furthermore, in other examples, the dynamic three-dimensional meridian system 100 of the human body can further include a voice playing device (not shown), the human meridian program 120 can further include a voice guide module (not shown). When the operating module 123 receives the animation playing instruction to display one of the meridian circulation video files on the touch-screen monitor 130, the voice guide module will output a voice playing signal to the voice playing device, and then the voice playing device will play a reading guide voice data based on the one of the meridian circulation video files. Therefore, it is favorable for clearly presenting the meridian circulation data for a better understanding, so that the dynamic and realistic visual presenting effects of the meridians along the order of the acupoints and based on the actual depths thereof can be obtained, and the needs of medical education and medical research can be satisfied.

Furthermore, the dynamic three-dimensional meridian system 100 of the human body of the present disclosure can further include the information about acupuncture and other related equipment, and the user can operate the touch-screen monitor 130 by tapping so as to select the menu to read the graphic knowledge of the acupuncture equipment, but the present disclosure is not limited thereto.

Therefore, the dynamic three-dimensional meridian system 100 of the human body of the present disclosure can present the circulation paths, the depths of acupoints and the change of the flowing in the meridians of the twelve primary meridians and the eight extraordinary vessels on the basis of the dynamic three-dimensional meridian image of the human body. Further, the human anatomy data as well as the acupoint location sites, indications, efficacy and other related clinical applications thereof can be integrated into the dynamic three-dimensional meridian system 100 of the human body of the present disclosure, so that it is favorable for the user to perform interactive operations so as to obtain the meridian names, the acupoint names and other information about the meridian circulation. Thus, the dynamic and realistic visual presenting effects of the meridians along the order of the acupoints and based on the actual depths thereof can be obtained, and the needs of medical education and medical research can be satisfied.

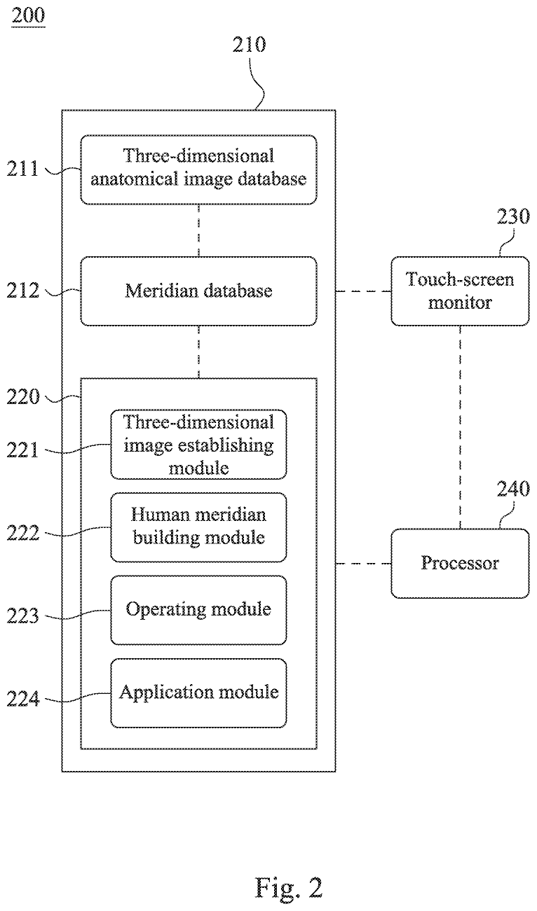

Please refer to FIG. 2, which is a block diagram of a dynamic three-dimensional meridian system 200 of a human body according to another embodiment of the present disclosure. The dynamic three-dimensional meridian system 200 of the human body includes a non-transitory machine readable medium 210, a touch-screen monitor 230 and a processor 240. The non-transitory machine readable medium 210 includes a human meridian program 220, a three-dimensional anatomical image database 211 of the human body and a meridian database 212. The three-dimensional anatomical image database 211 of the human body, the meridian database 212, the touch-screen monitor 230 and the processor 240 are respectively the same to the three-dimensional anatomical image database 111 of the human body, the meridian database 112, the touch-screen monitor 130 and the processor 140 of FIG. 1, so that the details thereof will not be described herein again.

In detail, in the dynamic three-dimensional meridian system 200 of the human body, the human meridian program 220 includes a three-dimensional image establishing module 221, a human meridian building module 222, an operating module 223 and an application module 224, wherein the three-dimensional image establishing module 221, the human meridian building module 222 and the operating module 223 are respectively the same to the three-dimensional image establishing module 121, the human meridian building module 122 and the operating module 123 of FIG. 1, so that the details thereof will not be described herein again.

The application module 224 is for adjusting the plurality of acupoint location information and the plurality of acupoint depth-location information based on a length between two lateral ridges of a second knuckle of a middle finger of a subject so as to output a plurality of personalized acupoint location information and a plurality of personalized acupoint depth-location information. In particular, the clinical methods to identify the locations of the acupoints not only include the method using the markers on the body surface to determine acupoints and the method of bone-degree acupoint determination, but also can be selected according to the ratio of the joint width or the lateral width of the finger (that is, “Proportional unit of the body measurement”). However, according to individual differences, the finger size of a normal person has a certain proportional relationship with other parts of the body thereof, so that the dynamic three-dimensional meridian system 200 of the human body of the present disclosure takes the length between two lateral ridges of the second knuckle of the middle finger being flexed of the subject as one inch to measure the distance between acupoints and the depth of each of the acupoints (that is, “Proportional unit of the body measurement with the middle finger”). Thus, it is favorable for personalized adjusting according to the body shape of different users, and the accuracy for identifying the acupoints of the dynamic three-dimensional meridian system 200 of the human body of the present disclosure can be enhanced. Furthermore, it must be noted that in addition to using the length between two lateral ridges of the second knuckle of the middle finger as a basis to determine the location of the acupoints, the dynamic three-dimensional meridian system 200 of the human body of the present disclosure also can take the distance between the two lateral ridges on the lateral edge of the middle segment of the middle finger being flexed as one inch, take the width of the tip of the thumb as one inch, or take other methods to facilitate the personalized adjustment, but the present disclosure is not limited thereto.

Furthermore, in other embodiments, the dynamic three-dimensional meridian system 200 of the human body can further include a diagnostic expert module, wherein the diagnostic expert module is for outputting diagnostic suggestion information for the subject based on the personalized acupoint location information and the personalized acupoint depth-location information. The diagnostic expert module of the present disclosure is established according to the acupoint location sites and the indications or diseases corresponding thereto disclosed in the book published by the lecturing professor Jaung-Geng Lin, and the diagnostic expert module can confirm the condition of the subject based on the personalized acupoint location information and the personalized acupoint depth-location information and then recommend suitable acupoints to the doctors. Preferably, the diagnostic expert module of the present disclosure can provide suggestions of the pathological mechanisms, the diagnosis, the treatment and the acupoints of the neurological system diseases, the motor system diseases, the respiratory system diseases, the cardiocirculatory system diseases, the gastrointestinal system diseases, the hepatobiliary system diseases, the genitourinary system diseases, the gynecological diseases, the facial diseases, the skin diseases, the pediatric diseases, etc., but the present disclosure is not limited thereto. Therefore, it is favorable for establishing the diagnosis and treatment information of important diseases and symptoms in various systems of the body of the subject, so that it has related application potential.

According to above, the dynamic three-dimensional meridian system 200 of the human body can present the circulation paths, the depths of acupoints and the change of the flowing in the meridians of the twelve primary meridians and the eight extraordinary vessels on the basis of the dynamic three-dimensional meridian image of the human body. Further, the human anatomy data, the acupoint location sites, indications, efficacy and other related clinical applications thereof as well as the personalized body information of the subject can be integrated into the dynamic three-dimensional meridian system 200 of the human body of the present disclosure. Therefore, the personalized acupoint depth-location information and the personalized meridian circulation data corresponding thereto can be obtained, so that the dynamic and realistic visual presenting effects of the meridians along the order of the acupoints and based on the actual depths thereof can be obtained, and the needs of medical education and medical research can be satisfied.

### Example

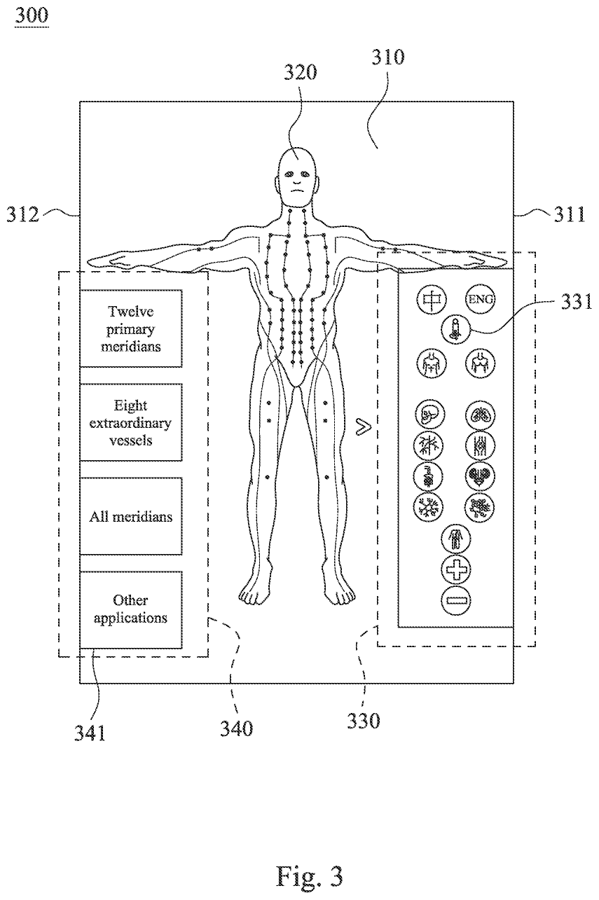

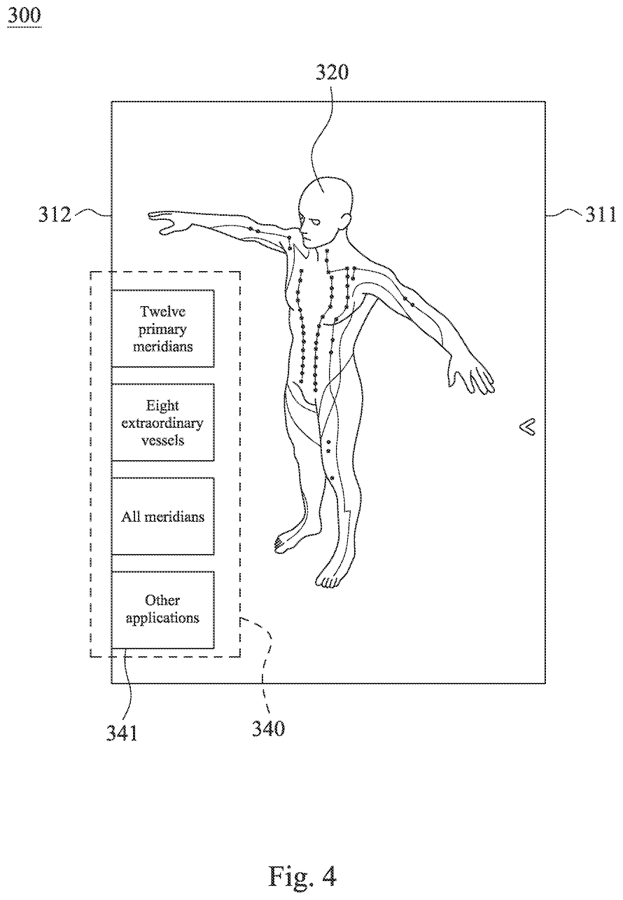

Please refer to FIG. 3 and FIG. 4. FIG. 3 is a schematic view of a dynamic three-dimensional meridian system 300 of a human body of the present disclosure, and FIG. 4 is another schematic view of the dynamic three-dimensional meridian system 300 of the human body of FIG. 3. The dynamic three-dimensional meridian system 300 of the human body includes a non-transitory machine readable medium (not shown), a touch-screen monitor 310 and a processor (not shown), wherein the dynamic three-dimensional meridian system 300 of the human body can be disposed in a computer, a smart phone or other electronic devices that can support the performance of the touch-screen monitor 310, but the present disclosure is not limited thereto.

As shown in FIG. 3 and FIG. 4, a dynamic three-dimensional meridian image 320 of the human body and a graphical user interface (reference number is omitted) of a human meridian program (not shown) are displayed on the touch-screen monitor 310, and the graphical user interface can include a plurality of touch icons 331 and a plurality of functional listings 341. In particular, the plurality of touch icons 331 can respectively correspond to options of a variety of different functions, and the touch icons 331 and the functional listings 341 can be respectively located on a first side portion 311 and a second side portion 312 of the touch-screen monitor 310, wherein the plurality of touch icons 331 can form a first interface arrangement unit 330, the plurality of functional listings 341 can form a second interface arrangement unit 340, and the first interface arrangement unit 330 and the second interface arrangement unit 340 can be displayed (as shown in FIG. 3) or hidden (as shown in FIG. 4) by being tapped and dragged left or right.

In the dynamic three-dimensional meridian system 300 of the human body, an operating module (not shown) of the human meridian program is for receiving an image operating instruction so as to adjust an image displaying condition of the dynamic three-dimensional meridian image 320 of the human body. In detail, when the user tapes the one of the touch icons 331 or performs to pinch open or closed with two fingers, the processor will output the image operating instruction to the operating module of the human meridian program simultaneously. The image operating instruction can include a zooming sub-instruction or an angle adjusting sub-instruction, and the image displaying condition can include an image displaying size and an image displaying angle, and the operating module adjusts the image displaying size and the image displaying angle of the dynamic three-dimensional meridian image 320 of the human body based on the zooming sub-instruction or the angle adjusting sub-instruction (as shown in FIG. 4).

As shown in FIG. 3 again, the touch icons 331 can respectively correspond to a bone composition information, an organ distribution information, a blood vessel distribution information, a lymphatic distribution information, a nerve distribution information and a skin condition information of the dynamic three-dimensional meridian image 320 of the human body of different gender. When the user tapes a corresponding one of the touch icons 331, the processor will output an image-presenting adjusting sub-instruction to the operating module of the human meridian program simultaneously. At this time, the operating module can show at least one of the bone composition information, the organ distribution information, the blood vessel distribution information, the lymphatic distribution information, the nerve distribution information and the skin condition information of the dynamic three-dimensional meridian image 320 of the human body based on the image-presenting adjusting sub-instruction, and the dynamic three-dimensional meridian image 320 of the human body of a male human body or the dynamic three-dimensional meridian image of the human body of a female human body (not shown) can be switched to each other according to actual needs.

In the dynamic three-dimensional meridian system 300 of the human body, the dynamic three-dimensional meridian image 320 of the human body includes a plurality of acupoint location information, a plurality of acupoint depth-location information and a plurality of meridian distribution information. The plurality of acupoint location information respectively correspond to a plurality of acupoint location sites which are shown on the dynamic three-dimensional meridian image 320 of the human body, and the plurality of meridian distribution information are established based on actual depths of acupoints of the plurality of acupoint data corresponding thereto and in a circulation order thereof. As shown in FIG. 3 again, the presentation of the meridian circulation data corresponding to the meridian distribution information can be operated by the plurality of functional listings 341. When the user tapes one of the functional listings 341 corresponding to the different meridian circulation data, the functional listing 341 will extend from the second side portion 312 and then displayed on the touch-screen monitor 310, and then the user can further tap the meridian circulation data which is wanted to show on the dynamic three-dimensional meridian image 320 of the human body. Thus, it is favorable for the user to confirm and identify the distribution of meridians and their corresponding acupoint location sites.

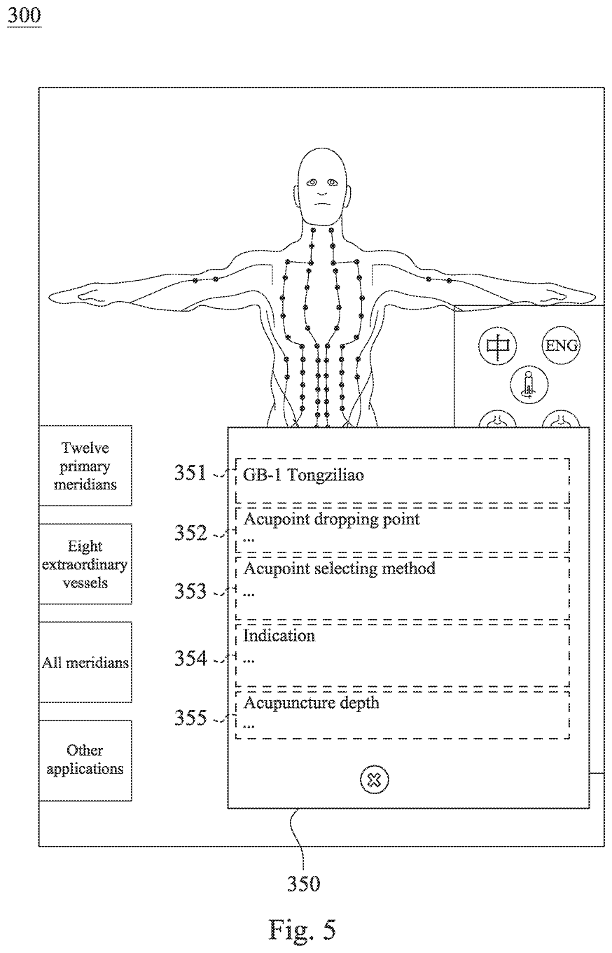

Please refer to FIG. 5, which is a schematic view of a touch-screen monitor 310 of the dynamic three-dimensional meridian system 300 of the human body of FIG. 3 for displaying the acupoint data 350. In detail, the acupoint data 350 includes an acupoint name information 351, an acupoint dropping point information 352, an acupoint selecting method information 353, an indication information 354 and an acupuncture depth information 355. When the user tapes a site of a specific acupoint of the dynamic three-dimensional meridian image 320 of the human body, the processor will output an acupoint presenting instruction to the operating module of the human meridian program simultaneously. At this time, the operating module can display one of the plurality of acupoint data 350 on the touch-screen monitor 310 based on the acupoint presenting instruction, so that it is favorable for the user to confirm the information of the acupoint data 350.

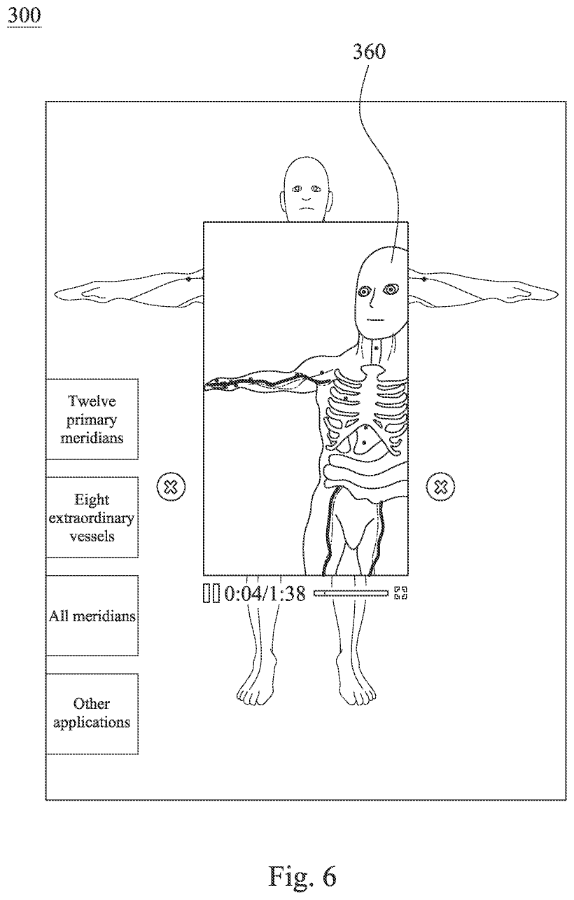

Please refer to FIG. 6, which is a schematic view of the touch-screen monitor 310 of the dynamic three-dimensional meridian system 300 of the human body of FIG. 3 for displaying a meridian circulation video file 360. In detail, the plurality of meridian circulation data of the dynamic three-dimensional meridian system 300 of the human body include a plurality of meridian circulation video files 360. When the user tapes one of the functional listings 341 including the meridian circulation data which is wanted to be shown, the processor will simultaneously output an animation playing instruction to the operating module of the human meridian program. At this time, the operating module will display one of the meridian circulation video files 360 on the touch-screen monitor 310 based on the animation playing instruction. At the same time, a voice guide module (not shown) of the dynamic three-dimensional meridian system 300 of the human body can play a reading guide voice data along with the playing of the meridian circulation video file. Therefore, it is favorable for dynamically displaying the paths and the distribution in the human body of the circulation and the flowing of the meridians.

Furthermore, other detailed information of the dynamic three-dimensional meridian system 300 of the human body are the same to that of the dynamic three-dimensional meridian system 100 of the human body and the dynamic three-dimensional meridian system 200 of the human body, so that the same details thereof are not described herewith again. Moreover, the human meridian program of the dynamic three-dimensional meridian system 300 of the human body can further include an application module so as to adjust the acupoint location information and the acupoint depth-location information of each of the acupoint based on a length between two lateral ridges of a second knuckle of a middle finger being flexed of a subject. Thus, the accuracy for identifying the acupoints of the dynamic three-dimensional meridian system 300 of the human body of the present disclosure can be enhanced, but the present disclosure is not limited thereto.

To sum up, the dynamic three-dimensional meridian system of the human body of the present disclosure can integrated the meridian circulation, acupoint location sites, acupoint depths, indications, efficacy and other related clinical applications thereof recorded on the traditional Chinese medicine there into, and then present on the simulated three-dimensional image of the human body. Thus, the dynamic and realistic visual presenting effects of the meridians along the order of the acupoints and based on the actual depths thereof can be obtained, and the needs of medical education and medical research can be satisfied. Furthermore, the dynamic three-dimensional meridian system of the human body of the present disclosure can further integrate the body information of the user so as to obtain the personalized acupoint location information, the personalized acupoint depth-location information and the personalized meridian circulation data. Thus, the personalized medical application can be satisfied under the premise of the dynamic and realistic visual presenting effects of the meridians along the order of the acupoints and based on the actual depths thereof, and it has related application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A dynamic three-dimensional meridian system of a human body, comprising:
a non-transitory machine readable medium comprising a human meridian program, a three-dimensional anatomical image database of the human body and a meridian database, wherein the three-dimensional anatomical image database of the human body comprises a plurality of three-dimensional anatomical image data of the human body, the meridian database comprises a plurality of acupoint data and a plurality of meridian circulation data, and the human meridian program comprises:
a three-dimensional image establishing module for processing the plurality of three-dimensional anatomical image data of the human body so as to obtain a simulated three-dimensional image of the human body;
a human meridian building module for integrating the plurality of acupoint data, the plurality of meridian circulation data and the simulated three-dimensional image of the human body so as to obtain a dynamic three-dimensional meridian image of the human body, wherein the dynamic three-dimensional meridian image of the human body comprises a plurality of acupoint location information, a plurality of acupoint depth-location information and a plurality of meridian distribution information; and
an operating module for receiving an image operating instruction so as to adjust an image displaying condition of the dynamic three-dimensional meridian image of the human body;
a touch-screen monitor signally connected to the non-transitory machine readable medium, wherein the touch-screen monitor is for displaying the dynamic three-dimensional meridian image of the human body and a graphical user interface of the human meridian program; and
a processor signally connected to the non-transitory machine readable medium and the touch-screen monitor, wherein the human meridian program is performed by the processor;
wherein the plurality of acupoint location information respectively correspond to a plurality of acupoint location sites which are shown on the dynamic three-dimensional meridian image of the human body, and the plurality of meridian distribution information are established based on actual depths of acupoints of the plurality of acupoint data corresponding thereto and in a circulation order thereof.

2. The dynamic three-dimensional meridian system of the human body of claim 1, wherein the plurality of meridian circulation data comprise a twelve primary meridians circulation data and an eight extraordinary vessels circulation data.

3. The dynamic three-dimensional meridian system of the human body of claim 2, wherein:
the plurality of meridian circulation data comprise a plurality of meridian circulation video files, and the operating module receives an animation playing instruction to display one of the plurality of meridian circulation video files on the touch-screen monitor; and
the one of the plurality of meridian circulation video files is displayed on the touch-screen monitor in a sub-window, and the plurality of acupoint location information, the plurality of acupoint depth-location information and the plurality of meridian distribution information are simultaneously displayed on the touch-screen monitor.

4. The dynamic three-dimensional meridian system of the human body of claim 3, wherein:
the dynamic three-dimensional meridian system further comprises a voice playing device, and the human meridian program further comprises a voice guide module; and
when the operating module receives the animation playing instruction to display the one of the plurality of meridian circulation video files on the touch-screen monitor, the voice guide module outputs a voice playing signal to the voice playing device, and then the voice playing device plays a reading guide voice data based on the one of the plurality of meridian circulation video files.

5. The dynamic three-dimensional meridian system of the human body of claim 1, wherein the image operating instruction comprises a zooming sub-instruction, an angle adjusting sub-instruction and an image contrast adjusting sub-instruction, the image displaying condition comprises an image displaying size, an image displaying angle and an image contrast, and the operating module adjusts the image displaying size, the image displaying angle or the image contrast of the dynamic three-dimensional meridian image of the human body based on the zooming sub-instruction, the angle adjusting sub-instruction or the image contrast adjusting sub-instruction.

6. The dynamic three-dimensional meridian system of the human body of claim 1, wherein the plurality of three-dimensional anatomical image data of the human body are a three-dimensional anatomical image data of a male human body or a three-dimensional anatomical image data of a female human body.

7. The dynamic three-dimensional meridian system of the human body of claim 6, wherein:
the dynamic three-dimensional meridian image of the human body further comprises a bone composition information, an organ distribution information, a blood vessel distribution information, a lymphatic distribution information, a nerve distribution information and a skin condition information; and
the image operating instruction comprises an image-presenting adjusting sub-instruction, and the operating module shows at least one of the bone composition information, the organ distribution information, the blood vessel distribution information, the lymphatic distribution information, the nerve distribution information and the skin condition information based on the image-presenting adjusting sub-instruction.

8. The dynamic three-dimensional meridian system of the human body of claim 1, wherein the graphical user interface comprises a plurality of touch icons and a plurality of functional listings, and the plurality of touch icons and the plurality of functional listings are respectively disposed on a first side portion and a second side portion of the touch-screen monitor.

9. The dynamic three-dimensional meridian system of the human body of claim 8, wherein the plurality of touch icons form a first interface arrangement unit, the plurality of functional listings form a second interface arrangement unit, and the first interface arrangement unit and the second interface arrangement unit are displayed or hidden by being dragged left or right.

10. The dynamic three-dimensional meridian system of the human body of claim 1, wherein:
each of the plurality of acupoint data comprises an acupoint name information, an acupoint dropping point information, an acupoint selecting method information, an indication information and an acupuncture depth information, and the operating module receives an acupoint presenting instruction to display one of the plurality of acupoint data on the touch-screen monitor; and
the human meridian program further comprises:
an application module for adjusting the plurality of acupoint location information and the plurality of acupoint depth-location information based on a length between two lateral ridges of a second knuckle of a middle finger of a subject so as to output a plurality of personalized acupoint location information and a plurality of personalized acupoint depth-location information.

11. The dynamic three-dimensional meridian system of the human body of claim 10, further comprising:
a diagnostic expert module for outputting a diagnostic suggestion information for the subject based on the plurality of personalized acupoint location information and the plurality of personalized acupoint depth-location information.
